SEQUENCE LISTING (2 sequences)

SEQ ID NO: 1 - DNA (other nucleic acid), 18 bp; single-stranded, unknown
ggcccaggaacaatggct

SEQ ID NO: 2 - DNA (other nucleic acid), 21 bp; single-stranded, unknown
ccctccccacctaactttgat

(19) United States
(12) Patent Application Publication
(10) Pub. No.: US 2003/0147871 A1
(43) Pub. Date: Aug. 7, 2003

(54) Method and compositions for inhibiting angiogenesis and treating cancer with IL-12 and IL-18

(75) Inventors: Giorgio Trinchieri, Wynnewood, PA (US); William M.F. Lee, Wynnewood, PA (US); Christina M. Coughlin, Philadelphia, PA (US)

Correspondence Address: HOWSON AND HOWSON, ONE SPRING HOUSE CORPORATION CENTER, BOX 457, 321 NORRISTOWN ROAD, SPRING HOUSE, PA, 19477, US

(73) Assignee: The Trustees of the University of Pennsylvania, Philadelphia, PA

(21) Appl. No.: 10/353,283

(22) Filed: Jan. 29, 2003

Related U.S. Application Data
(63) Continuation of application No. 08/963,060, filed on Nov. 3, 1997.

(51) Int. Cl. A61K 48/00

(52) U.S. Cl. 424/93.21

(57) ABSTRACT

A composition useful for preventing, or retarding the growth of, tumor cells contains synergistic amounts of Interleukin-12 and Interleukin-18. Similarly, methods for treating or preventing cancer include co-administering synergistic amounts of IL-12 and IL-18. The resulting anti-tumor effect is greater than the additive effect of either cytokine administered alone.

### CROSS-REFERENCE TO RELATED APPLICATIONS

This application is a continuation of U.S. patent application Ser. No. 08/963,060, filed Nov. 3, 1997.

This invention has been supported by grants from the National Institutes of Health, Grant Nos. HL02939, CA20833 and AI34412. The United States government has an interest in this invention.

### BACKGROUND OF THE INVENTION

Tumor cell immunization is a vaccine option in the treatment and prophylaxis of cancers, and generally offers an advantage in cancer therapy because all potential tumor antigens are present in the vaccine composition. However, in certain cancers, specific tumor antigens have not been identified. Thus, an alternative strategy for tumor immunotherapy of these cancers attempts to enhance host responses to tumor antigens by creating a local environment favorable for antigen presentation and immunological recognition of tumor cells.

To accomplish this, tumor cells have been engineered to express immunostimulatory cytokines, such as interleukin-12 (IL-12) (A. Martinotti et al, 1995 Eur. J. Immunol., 25:137-146; M. Colombo et al, 1996 Cancer Res., 56:2531-2534; and H. Tahara et al, 1995 J. Immunol., 154:6466-6474). Several different murine tumors engineered to express mIL-12 induce enhanced cell-mediated immunity and tumor-specific rejection responses in syngeneic mice (A. Martinotti et al, cited above, H. Tahara et al, cited above; and T. Wynn et al, 1995 J. Immunol., 154:3999-4009). Nonmalignant fibroblasts engineered to secrete mIL-12 have been injected with native tumor cells to achieve similar results, presumably through “paracrine” activities of the cytokine-(H. Tahara et al, 1994 Cancer Res., 54:182-189). Recombinant IL-12 administered to A/J mice injected with SCK mammary carcinoma cells delays, but does not prevent tumor development (see, C. Coughlin et al, cited above).

How IL-12 achieves these antitumor effects is less certain, in part because its pleiotropic effects make it difficult to ascribe anti-tumor effectiveness to any individual mechanism. IL-12 favorably alters the host-tumor relationship through any of several direct and indirect effects on lymphoid and non-lymphoid cells (G. Trinchieri, 1995 Annu. Rev. Immunol., 13:251-276). It enhances cellular immune mechanisms by favoring the differentiation of CD4⁺ helper T cells towards the T_{H}1 subset (X. Gao et al, 1989 J. Immunol., 143:3007-3014). T_{H}1 cells secrete IL-2 and interferon-γ (IFN-γ) which are cytokines that facilitate the proliferation and/or activation of CD8⁺ cytolytic T cells (CTLs), natural killer (NK) cells and macrophages, all of which can contribute to tumor regression (E. Bloom et al, 1994 J. Immunol., 152:4242-4254; C. Nastala et al, 1994 J. Immunol., 153:1697-1706; and K. Tsung et al, 1997 J. Immunol., 158:3359-3365).

Many of IL-12's effects, both therapeutic and toxic, have been shown to depend on the IL-12-stimulated IFN-γ secretion by T and NK cells (C. Nastala et al, cited above; C. Coughlin et al, 1995 Cancer Res., 55:4980-4987; C. Coughlin et al, 1997 Cancer Res., 57:2460-2467; and B. Car et al, 1995 Am. J. Path., 147:1693-1707). Other responses to IL-12 may be due to the TNF-α that is also induced (C.

Biron et al, 1995 Res. Immunol., 62:590-600 and J. Orange et al, 1994 J. Immunol., 152:1253-1264). IFN-γ alters tumor cell behavior and host responses to tumor cells in a variety of ways, many of which favor tumor regression. These include a slowing of cellular proliferation (U. Boehm et al, 1997 Annu. Rev. Immunol, 15:749-795), upregulation of tumor cell MHC expression (K. Tsung et al, cited above; and W. Yu et al, 1996 Int. Immunol., 8:855-865), induction of nitric oxide production (M. Revel et al, 1986 Trends Biochem. Sci., 11:166-170 and Q. Xie et al, 1993 J. Exp. Med., 177:1779-1784) and inhibition of angiogenesis, which has received particular attention (E. Voerst et al, 1995 J. Natl. Cancer Inst., 87:581-586 and C. Sgadari et al, 1996 Blood, 87:3877-3882). Many of the proposed mechanisms of the antitumor activity of IL-12 do not rely on antigen specific immune response, but rely instead on innate, non-specific responses of the immune system.

There remains a need in the art for additional methods and compositions which are useful in preventing or retarding the growth of tumors generally, and particularly those for which tumor antigens have yet to be identified.

### SUMMARY OF THE INVENTION

The present invention relates generally to compositions and methods of use thereof in inhibiting the growth of tumor cells, and in anti-angiogenesis generally.

More specifically, the invention provides therapeutic compositions and methods for the treatment of cancer.

The present invention addresses the need in the art by providing compositions containing both IL-12 and IL-18, and methods for administering both cytokines together to achieve a synergistic effect. Although each cytokine alone has anti-tumor effects, it was unpredictable prior to the present invention that administered together, the effect of IL-12 and IL-18 was synergistic and could provoke not only a protective immune (i.e, antitumor) response, but an effective, systemic response in treated mammals. Thus, in one aspect, the invention provides a composition useful for killing, or retarding the growth, of tumor cells comprising:

(a) an effective amount of Interleukin-12, or a fragment thereof which has the biological function of said IL-12; and

(b) an effective amount of Interleukin-18 or a fragment thereof which has the biological function of said IL-18.

In another aspect, the invention provides a therapeutic method for retarding the growth of a tumor comprising administering to a mammal with said tumor an effective amount of the composition above.

In yet another aspect, the invention provides a method for preventing the growth of a tumor comprising administering to a mammal with said tumor an effective amount of the composition above.

In still a further aspect, the invention provides a method for providing systemic protection against the growth of tumor cells comprising administering to a mammal in need thereof a synergistic amount of IL-12 and IL-18.

In yet a further aspect, the invention provides an improved method for the treatment of a cancer which comprises the administration of IL-12, in which the improvement comprises concurrently administering a synergistic amount of IL-18.

Still another aspect of the invention is an improved method for the treatment of a cancer which comprises the administration of IL-18, the improvement comprising concurrently administering a synergistic amount of IL-12.

Other aspects and advantages of the present invention are described further in the following detailed description of the preferred embodiments thereof

### BRIEF DESCRIPTION OF THE DRAWINGS

FIG. 1A is a graph plotting tumor development in A/J mice (% with tumors) receiving SCK murine mammary carcinoma cells expressing mIL-12 (SCK.12C) or mIL-18 (SCK.18A) as described in Example 3 or 4 vs. days post tumor cell injection. Black, solid lines represent tumorigenesis in mice injected with SCK cells; grey, solid lines represent tumorigenesis in mice injected with SCK.18A cells; black, dashed lines represent tumorigenesis in mice injected with SCK.12C cells. These data are from a single, representative experiment.

FIG. 1B is a graph similar to that of FIG. 1A, except that the mice were severe combined immunodeficient (SCID) mice. All symbols and procedures were otherwise as described in FIG. 1A.

FIG. 2A is a histological photomicrograph analysis of an SCK tumor at 20× magnification. See, Example 5.

FIG. 2B is a histological photomicrograph analysis of an SCK tumor at 60× magnification in A/J mice treated as described in FIG. 2A. Arrows indicate individual cells undergoing cell death.

FIG. 2C is a histological photomicrograph analysis of SCK.12C tumor at 10× magnification. A/J mice were injected with 1×10⁶ SCK.12C cells, and otherwise treated as in FIG. 2A. Arrow indicates the area of viable tumor cells.

FIG. 2D is a histological photomicrograph analysis of SCK.12C tumor at 60× magnification. Arrows indicate areas of infiltration. Mice were treated as in FIG. 2C.

FIG. 2E is a histological photomicrograph analysis of SCK.18A tumor at 20× magnification. A/J mice were injected with 2.5×10⁴ SCK.18A cells, and otherwise treated as in FIG. 2A. Arrows indicate focal areas of necrosis.

FIG. 2F is a histological photomicrograph analysis of SCK.18A tumor at 60× magnification. Arrows indicate areas of infiltration. Mice were treated as in FIG. 2E.

FIG. 3A is a graph plotting SCK tumor development in A/J mice (% with tumors) injected with SCK and SCK.12C cells vs. days post tumor cell injection.

Symbols are as follows: mice receiving SCK cells alone (black solid lines); mice receiving SCK.12C and SCK cells co-injected in one flank (“ipsi”; black, dashed lines); mice receiving SCK and SCK.12C cells in opposite flanks (“contra”; grey lines).

FIG. 3B is a graph plotting SCK tumor development in A/J mice (% with tumors) injected with SCK and SCK.18A cells vs. days post tumor cell injection. Mice that received 2.5×10⁴ SCK cells alone are represented by black solid lines; mice that received SCK.18A and SCK cells co-injected in one flank by “ipsi”; black, dashed lines; and mice that received SCK and SCK.18A cells in opposite flanks by “contra”; grey lines.

FIG. 3C is a graph plotting SCK tumor development in A/J mice (% with tumors) injected with SCK and SCK.18A+SCK.12C cells vs. days post tumor cell injection. Symbols are: mice that received SCK cells alone (black solid lines); and mice that received co-injections of SCK.12C+SCK.18A cells with these cells in the flank opposite the SCK cells (grey lines).

FIG. 4A is a photograph of Matrigel® implants containing no cells harvested from A/J mice on day 4.

FIG. 4B is a photograph of a Matrigel® implants containing SCK cells harvested from A/J mice on day 4. FIG. 4C is a photograph of a Matrigel® implants containing SCK.12C cells harvested from A/J mice on day 4.

FIG. 4D is a photograph of a Matrigel® implants containing SCK.18A cells harvested from A/J mice on day 4.

FIG. 5A is a graph showing hemoglobin content of Matrigel® implants containing no cells, SCK, SCK.12C or SCK.18A cells.

FIG. 5B is a graph showing hemoglobin content of Matrigel® implants containing no cells or only SCK cells. SCK.12C, SCK.18A or both types of cells were injected into animals which received the Matrigel® implants with SCK cells at a distant site.

FIG. 5C is a graph showing hemoglobin content of Matrigel® implants containing no cells or only SCK cells. Some mice having the Matrigel® implants containing SCK cells were injected with SCK.12C+SCK.18A cells at a distant site, and some of these were treated with anti-IFN-γ monoclonal antibody (mAb) on days −1, 0 and 3. FIG. 5D is a graph showing hemoglobin content of Matrigel® implants containing no cells, SCK cells (1×10⁵), C1300 cells (1×10⁶) or Sa-1 cells (1×10⁶) or 10 ng recombinant bovine fibroblast growth factor (rb-FGF). Half of the mice in each group were injected with SCK.12C+SCK.18A cells at a distant site. (*) and (+) indicate groups with significantly different hemoglobin content (p<0.05). Each group contained three mice whose implants were assayed separately. Bars indicate the standard deviation of the hemoglobin measurements.

### DETAILED DESCRIPTION OF THE INVENTION

The invention provides a synergistic composition of two cytokines and methods of using such compositions for killing, or retarding the growth of, tumor cells in a mammal. Thus, such methods may be both a therapeutic and prophylactic treatment for cancers.

I. Composition of the Invention

The composition of the present invention comprises an effective amount of Interleukin-12, or a fragment thereof which has the biological function of IL-12, and an effective amount of Interleukin-18 or a fragment thereof which has the biological function of IL-18. While each cytokine alone has measurable antitumor effects, the two together are necessary to induce a protective, systemic antitumor response. The antitumor effects of the combination of IL-18 and IL-12 depend in large part on gamma interferon (IFN-γ). Examination of the mechanisms of protection indicated that induction of antitumor immunity was hastened and inhibition of tumor angiogenesis was enhanced by the combination of the two cytokines, suggesting that these antitumor activities of IL-12+IL-18 contribute to the synergistic therapeutic effectiveness of these two cytokines against tumors. Thus, the composition is characterized by the ability to inhibit angiogenesis, and such inhibition is likely responsible for the synergistic antitumor effect produced by the combination of IL-12 and IL-18.

A. Interleukin-12

Interleukin-12 (IL-12), originally called natural killer cell stimulatory factor, is a heterodimeric cytokine described, for example, in M. Kobayashi el al, 1989 J. Exp. Med., 1709:827. The expression and isolation of IL-12 protein in recombinant host cells is described in detail in International Patent Application WO90/05147, published May 17, 1990 (also European patent application No. 441,900), incorporated by reference herein. The DNA and amino acid sequences of the 30 kd and 40 kd subunits of the heterodimeric human IL-12 are provided in the above recited international application, and are reproduced in the Sequence Listing attached hereto.

Research quantities of recombinant human and murine IL-12 are also available from Genetics Institute, Inc., Cambridge, Mass. IL-12 has been found to stimulate IFN-γ production by NK cells and T cells (S. H. Chan et al, 1991 J. Exp. Med., 173:869). Therapeutic effects of IL-12 administered systemically have been reported (See, e.g., F. P. Heinzel el al, 1993 J. Exp. Med., 177:1505 and J. P. Sypek et al, ibid, p. 1797, among others). Where it is used throughout the examples, the term IL-12 refers to the heterodimeric protein unless smaller fragments thereof are specifically identified. Fragments of IL-12 which share the same biological activity of the full-length protein as well as the DNA sequences which encode IL-12 or fragments thereof may also be employed as the IL-12 of the compositions. Such biologically active fragments may be obtained by conventional recombinant engineering methods of fragmenting a protein. Any fragment may be readily assessed for IL-12 biological activity by testing in an assay which measures the induction of interferon-γ secretion by human lymphocytes (M. Wysocka et al, 1995 Eur. J. Immunol., 25:672-676). It should be understood by one of skill in the art, that such identification of suitable biologically active fragments of IL-12 for use in the composition of this invention involves only a minor amount of routine experimentation.

B. Interleukin-18

Interleukin-18 (IL-18) is a recently identified cytokine which induces IFN-γ release (S. Ushio et al, 1996 J. Immunol., 156:4274-4279), also called interferon-γ-inducing factor (IGIF) (H. Okamura et al, 1995 Nature, 378:88-91). The latter reference provides the coding sequence of IGIF and is incorporated herein by reference. IL-18 is produced by Kupffer cells and activated macrophages, promotes IFN-γ release (M. Micallef et al, 1996 Eur. J. Immunol., 26:1647-1651) and inhibits the production of IL-10 by activated T cells (S. Ushio et al, cited above and H.

Okamura et al, cited above). IL-18 augments both murine and human NK cytotoxicity (S. Ushio et al, cited above and H. Okamura et al, cited above) and stimulates Fas ligand-mediated tumor cell cytotoxicity by NK cells (H. Tsutsui et al, 1996 J. Immunol., 157:3967-3973). While IL-18 responses resemble those of IL-12 (particularly in promoting cellular immune responses and T cell release of IFN-γ), the two cytokines do not have identical effects inasmuch as they synergistically induce T cell production of IFN-γ in vitro (M. Micallef et al, cited above). The described activities of IL-18 suggest that it might have antitumor activity. Recently, administration of recombinant mIL-18 was shown to enhance the survival of BALB/c mice bearing Meth A tumors (M. Micallef et al, 1997 Cancer Immunol. Immunother., 43:361-367).

Fragments of IL-18 which share the same biological activity of the full-length protein as well as the DNA sequences which encode IL-18 or fragments thereof may also be employed as the IL-18 of the compositions. Such biologically active fragments may be obtained by conventional recombinant engineering methods of fragmenting a protein. Any fragment may be readily assessed for IL-18 biological activity by testing in the assay for the stimulation of interferon-γ induction in synergy with IL-12 (Wysocka et al, cited above). It should be understood by one of skill in the art, that such identification of suitable biologically active fragments of IL-18 for use in the composition of this invention involves only a minor amount of routine experimentation.

C. Therapeutic Compositions of the Invention

The composition containing IL-12 and IL-18 of the present invention may be prepared in any suitable form for administration to a mammal. For example, the IL-12 and IL-18 components of the compositions may be in the form of full-length proteins, or peptide fragments thereof as discussed above. Such proteins or peptides may be purchased commercially, or may be generated by well-known standard recombinant engineering or chemical synthetic techniques (i.e., transfected into and expressed by a host cell, and isolated therefrom) based on the known coding sequences thereof. See, for example, the techniques disclosed in International Patent Application WO90/05147, cited above.

When the components are in the form of protein, they may be administered in a suitable pharmaceutical formulation with optional conventional pharmaceutical carriers, such as phosphate buffered saline, or pH stabilizers, and the like. Preparation of such a proteinaceous pharmaceutical composition is conventional and involves merely mixing the IL-12 and IL-18 components with the selected optional pharmaceutical additives.

Alternatively, it is also anticipated that nucleic acid sequences encoding IL-12 or a fragment thereof and IL-18 or a fragment thereof may be used as a pharmaceutical composition of the invention. The nucleic acid sequences, preferably in the form of DNA, may be delivered to provide for in vivo expression of the IL-12 and IL-18 proteins or peptides. Delivery of a protein in the form of ‘naked DNA’ is within the skill of the art. (See, e.g., J. Cohen, Science, 259:1691-1692 (Mar. 19, 1993); E. Fynan et al, Proc. Natl. Acad. Sci., 90: 1478-11482 (December 1993); J. A. Wolff et al, 1991 Biotechniques, 11:474-485, which describe similar uses of ‘naked DNA’, all incorporated by reference herein).

As another alternative, the IL-12 and IL-18 DNA may be incorporated, or transduced, into a DNA molecule, i.e., a plasmid vector, of which many types are known, or into a viral vector, preferably a poxvirus vector or adenovirus vector, for delivery of the IL-12 and IL-18 DNA into the patient. When incorporated into another DNA molecule, the DNA sequence encoding the IL-12 or IL-18 is operatively linked with regulatory sequences which direct the expression of the encoded protein or fragment in vivo. Briefly, a cassette may be engineered to contain, in addition to the IL-12 and/or IL-18 sequence to be expressed, other flanking sequences which enable insertion into a vector. This cassette may then be inserted into an appropriate DNA vector downstream of a promoter, an mRNA leader sequence, an initiation site and other regulatory sequences capable of directing the replication and expression of the desired IL-12 and/or IL-18 sequence in a host cell. When administered as naked DNA or as part of plasmid or viral vectors, the sequences encoding IL-12 and IL-18 may be present on separate DNA molecules which are admixed for administration, or may be assembled as part of a single polycistronic molecule, under the control of the same or different regulatory sequences.

Numerous types of appropriate vectors are known in the art for protein expression and may be designed by standard molecular biology techniques. Such vectors are selected from among conventional vector types including insects, e.g., baculovirus expression, or yeast, fungal, bacterial or viral expression systems. Methods for obtaining such vectors are well-known. See, Sambrook et al, Molecular Cloning. A Laboratory Manual, 2d edition, 1989 Cold Spring Harbor Laboratory, New York; Miller et al, 1986 Genetic Engineering, 8:277-298 (Plenum Press) and references cited therein. Recombinant viral vectors, such as retroviruses or adenoviruses, are preferred for integrating the exogenous DNA into the chromosome of the cell.

Also where desired, the regulatory sequences in such a vector which controls and directs expression of the IL-12 and/or IL-18 gene product in the transfected cell includes an inducible promoter. Inducible promoters are those which “turn on” expression of the gene when in the presence of an inducing agent. Examples of suitable inducible promoters include, without limitation, the sheep metallothionine (MT) promoter, the mouse mammary tumor virus (MMTV), the tet promoter, etc.

The inducing agents may be a glucocorticoid such as dexamethasone, for, e.g., the MMTV promoter, or a metal, e.g., zinc, for the MT promoter; or an antibiotic, such as tetracycline for tet promoter. Still other inducible promoters may be selected by one of skill in the art, such as those identified in International patent application WO95/13392, published May 18, 1995, and incorporated by reference herein. The identity of the inducible promoter is not a limitation of this invention.

Therapeutic compositions of this invention may be formulated to contain the IL-12 and IL-18 as proteins or DNA, along with a carrier or diluent. Suitable pharmaceutically acceptable carriers facilitate administration of proteins or chemical compounds but are physiologically inert and/or nonharmful. Carriers may be selected by one of skill in the art. Exemplary carriers include sterile saline, lactose, sucrose, calcium phosphate, gelatin, dextrin, agar, pectin, peanut oil, olive oil, sesame oil, and water. Additionally, the carrier or diluent may include a time delay material, such as glycerol monostearate or glycerol distearate alone or with a wax. In addition, slow release polymer formulations can be used. Optionally, this composition may also contain conventional pharmaceutical ingredients, such as preservatives, or chemical stabilizers.

Alternatively, or in addition to the compounds of the invention, other agents useful in treating cancer, or useful in treating any accompanying bacterial or viral infection, e.g., antivirals, or immunostimulatory agents and cytokine regulation elements, or costimulatory molecules, such as B7, are expected to be useful in the compositions of this invention. Such agents may operate in concert with the therapeutic compositions of this invention and may be delivered to the patient as DNA or protein, or as a conventional pharmaceutical synthetic agent. The development of therapeutic compositions containing these agents is within the skill of one in the art in view of the teachings of this invention.

II. Methods of the Invention

Other aspects of this invention involve methods for the use of the compositions described above in therapeutic or vaccine regiments for the treatment or prophylaxis of cancer. More specifically, the present invention is useful when directed against those tumors for which antigens have yet to be identified.

Thus, the invention provides a therapeutic method for retarding or preventing the growth of a tumor comprising administering to a mammal with said tumor an effective amount of an IL-12 and IL-18 containing composition, such as described above. The methods of the invention also provide for systemic protection against the growth of tumor cells comprising administering to a mammal in need thereof a synergistic amount of IL-12 and IL-18.

The modes of administration may include administration of the cytokines as soluble proteins, or fragments thereof in a suitable pharmaceutical carrier; administration of DNA sequences encoding the cytokines which sequences are carried by viral or plasmid vectors injected into the subject; administration of viral or plasmid vectors carrying the sequences encoding the cytokine(s) ex vivo into a subject's tissue and readministration of the tissue, e.g., blood, fibroblasts, into the patient; administration of naked DNA encompassing the DNA sequences encoding the cytokine(s) via a genegun or other apparatus; intramuscular administration of plasmid based vectors; administration of transfected fibroblasts, etc.

Another alternative method would involve concurrently administering synergistic amounts of IL-18 and IL-12 in two separate compositions. Concurrent administration should be understood to include administering IL-18 or IL-12-containing compositions substantially simultaneously, or administering one compositions before the other.

According to these methods of the invention, a human or an animal may be treated for cancer by administering an “effective amount” of a therapeutic composition containing both IL-12 and IL-18, or a “synergistic amount” of individual compositions, one containing IL-12 and the other containing IL-18. Suitable “effective or synergistic amount” determinations may be made by the attending physician or veterinarian depending upon the age, sex, weight and general health of the human or animal patient and the cancer itself.

In one embodiment, the effective or synergistic amounts of IL-12 or IL-18 protein are between about 0.1 μg to about 0.5 mg of each protein. The IL-12 and IL-18 containing composition or each separate composition containing IL-12 or IL-18 is administered parenterally, preferably intramuscularly or subcutaneously. However, it may also be formulated to be administered by any other suitable route, including orally or topically.

In another embodiment, when administered as naked DNA or in plasmids, an effective or synergistic amount of a composition containing both IL-12 and IL-18 coding sequences, or separate compositions providing the IL-12 DNA and IL-18 DNA individually are amounts of DNA that will permit the in vivo production of between about 0.1 μg to about 0.5 mg of each protein.

In yet another embodiment, when administered via a viral vector, the amount of vector administered is sufficient plaque forming units to enable an infected cell to produce between about 0.1 μg to about 0.5 mg of each protein.

In general, dosages up to maximally tolerated dosages of both cytokines may be employed, and will ultimately be determined by the attending physician. Such effective dosages will be determined based on the cancer being treated, as well as the parameters normally used to determine pharmaceutical dosages listed above. Typically, the dosage of one cytokine may be modified if it is administered with the second cytokine or with an alternative additional agent, such as B7, as listed above. When IL-12 and IL-18 are administered together, lower dosages of each may be employed than when either cytokine is administered singly. Lower dosages of each cytokine when administered together may reduce toxic effects, such as possible transient immunosuppressive effects of IL-12. As may be further determined by the attending physician, repeated high dose administration of the combined composition may be desirable to treat certain resistant cancers. The determination of the dosages of each of these embodiments is within the skill of the art.

The examples below demonstrate the synergistic systemic antitumor effect and mechanisms activated by administration of a combination of IL-12 and IL-18, cytokines that induce IFN-γ production in vitro and in vivo. Briefly, these cytokines were secreted by genetically engineered SCK murine mammary carcinoma cells, and the effect upon the growth of the SCK carcinoma cells observed. Each cytokine alone retarded the tumorigenicity of the SCK cells; however surprisingly when both cytokines were administered together, a dramatically increased, and systemic tumoricidal effect was produced.

More specifically, using SCK murine mammary carcinoma cells as the tumor model, both SCK.12 and SCK.18 cells exhibited a reduction in tumorigenicity that correlated with the level of cytokine secreted. The effect is most striking in the case of SCK cells secreting mIL-12 where those secreting the most, SCK.12C cells, failed to form progressive tumors even when 40 times the usual tumorigenic dose of SCK cells was injected into syngeneic mice. These cells create an environment unfavorable for progressive tumor growth shown by their ability to prevent tumor formation by colocalized SCK cells. This effect is more local than systemic, however, and these cells only weakly prevent distant SCK cells from forming progressive tumors. Poor systemic protection speaks against antitumor immunity being the primary mechanism underlying rejection of SCK.12C tumors, and this conclusion is buttressed by the fact that only half of the mice injected with SCK.12C cells develop protective immunity and this immunity takes more than two weeks to become fully manifest. Nevertheless, antitumor immunity may be necessary for ultimate SCK.12C tumor rejection, because its absence in SCID mice resulted in progressive, fatal SCK.12C tumors.

Another candidate for the SCK.12C-activated mechanism that is principally responsible for tumor rejection is inhibition of tumor angiogenesis. These cells clearly inhibit angiogenesis, evidenced by their ability to inhibit vascularization of Matrigel® implants containing SCK cells. That antiangiogenesis is an important SCK.12C-activated antitumor mechanism is supported by the fact that neovascularization is necessary and limiting for tumor progression (N. Weidner et al, 1996 Imp. Adv. Onc., 8:167-190 and J. Folkman, 1997 EXS, 79:1-8) and by the fact that IFN-γ is important both for SCK.12C antitumor effects and for mIL-12 antiangiogenic effects (C. Sgadari et al, cited above). However, IFN-γ has pleiotropic activities many of which retard tumor growth or aid host removal of tumor cells (U. Boehm et al, cited above), and angiogenesis inhibition may not be the only IFN-γ activity of potential importance for SCK.12C antitumor effects.

The antitumor effects of SCK.18 cells were less striking than those of SCK.12C cells. Tumorigenesis by SCK.18 cells was reduced but not ablated, and these cells provided little if any protection against tumor formation by neighboring or distant SCK cells. However, SCK cells expressing higher levels of mIL-18 were unavailable for study. Thus, these results do not enable a direct comparison of the intrinsic antitumor effectiveness of mIL-18 and mIL-12. mIL-18 clearly does not need mIL-12 for its anti-tumor effects, because mIL-12-neutralization does not diminish its effectiveness.

It is theorized that these two cytokines activate many of the same antitumor mechanisms because both induce IFN-γ production by lymphocytes, and SCK.18 cells, like SCK.12 cells, require IFN-γ for their antitumor effects. This prediction was confirmed in the case of angiogenesis inhibition, since SCK.18A cells, like SCK.12C cells, induced markedly less Matrigel® implants neovascularization than SCK cells, and both SCK.18 and SCK.12 inhibit angiogenesis induced by SCK cells. This similarity suggests that angiogenesis inhibition may account for some of the shared features of SCK.18 and SCK.12 tumorigenesis, such as the IFN-y-dependent delay in tumor appearance.

Factors other than angiogenesis inhibition may account for the differences in the properties of SCK.12C and SCK.18A cells, such as the incidence of progressive tumors and their ability to inhibit tumor formation by colocalized SCK cells. Therefore, although both mIL-12 and mIL-18 induce endogenous production of IFN-γ, require IFN-γ for tumor regression and inhibit angiogenesis, the mechanisms responsible for tumor cell killing and tumor regression in SCK.12 and SCK.18 tumors may not be the same, and these differences may account for the varying antitumor effectiveness of SCK.12C and SCK.18 cells.

However, together, SCK.12 and SCK.18 cells induce greater antitumor effects than either cell type alone. This is most evident in the ability of the combination of the two cell types to cooperatively protect against SCK tumorigenesis systemically. However, the cooperative antitumor effect of combined mIL-12 and mIL-18 secretion is also evident by other parameters, such as greater inhibition of SCK induced angiogenesis, earlier induction of protective immunity and a greater delay in SCK tumor appearance. The mechanisms underlying cooperative induction of systemic protection by mIL-12+mIL-18 are unclear. Greater IFN-γ production may underlie this antitumor effect, because they can synergistically induce T cell production of IFN-γ in vitro (M. Micallef et al, cited above) and, together, can induce B cells to produce IFN-γ in vitro (T. Yoshimoto et al, 1997 Proc. Natl Acad. Sci., 94:3948-3953). However, serum IFN-γ levels in mice injected with SCK.12C cells, SCK.18A cells or both cell types have not been measured, and therefore cannot document this effect in vivo.

The development of protective immunity in only half of the mice rejecting SCK.12C and/or SCK.18A cells suggests that mechanisms other than antigen-specific immune responses are active. Others have also observed the absence of protective immunity in a significant percentage of survivors of mIL-12-secreting tumor cells (F. Cavallo et al, 1997 J. Natl. Cancer Inst., 89:1049-1058), and our data establish the same phenomenon in survivors of mIL-18-secreting tumor cells. Recombinant mIL-12 has potent antiangiogenic activity (E. Voerst et al, cited above and C. Sgadari et al, cited above). The levels of mIL-12 secreted by the engineered tumor cells of the examples has similar activity. Given the importance of angiogenesis for tumor growth, its inhibition is likely to be a crucial mechanism for the antitumor efficacy of secreted mIL-12.

It is presently theorized that mIL-12 acts via induction of IFN-γ production which, in turn, induces IP-10 production which is necessary for angiogenesis inhibition by mIL-12 (C. Sgadari el al, cited above and A. Angiolillo et al, 1995 J. Exp. Med., 182:155-162). mIL-18 may also inhibit angiogenesis through the induction of IFN-γ and IP-10. The following examples indicate that angiogenesis inhibition by mIL-12 and mIL-18 is an important component of their antitumor activity. Inhibition of angiogenesis is detectable early after injection of cells secreting mIL-12 and/or mIL-18 or after administration of rmIL-12 (unpublished observations) and probably accounts for their IFN-γ-dependent delay of tumorigenesis. That this is the principal antigen-nonspecific protective mechanism activated by SCK.12 or the combination of SCK.12 and SCK.18 cells is shown by activity against unrelated syngeneic tumors, such as Sa-1 sarcoma cells. However, angiogenesis inhibition may not be enough for ultimate protection from tumors.

Perhaps the appropriate perspective on angiogenesis inhibition during mIL-12-induced tumor regression is provided by studies of other angiogenesis inhibitors, such as angiostatin (M. O'Reilly et al, 1994 Cell, 79:315-328) and endostatin (M. O'Reilly et al, 1997 Cell, 88:277-285), which show that the most effective of these compounds induce shrinkage of large tumors and prevent growth of small tumors but do not eradicate residual tumor cells. If stasis of small tumor cell nests is the therapeutic limit of angiogenesis inhibitors, cure of SCK tumors by mIL-12 and mIL-18 is evidence that these cytokines invoke additional antitumor mechanisms and may be a compelling argument for bringing more than one antitumor mechanism to bear in order to overcome the limitations of any single mechanism.

The following examples thus illustrate various aspects of this invention and do not limit the invention, the scope of which is embodied in the appended claims.

### EXAMPLE 1

### Construction of Cytokine-Expressing Tumor Cells

The SCK mammary carcinoma cell line (gift from Dr. J. G. Rhee, University of Md., Baltimore, Md.) (C. Song et al, 1994 Br. J Cancer, 41:309-312) was derived from a tumor that spontaneously arose in an A/J mouse (H-2a) and is maintained in RPMI medium supplemented with 10% FCS and penicillin/streptomycin.

A. Cells Expressing IL-12

To create SCK cells that express murine IL-12 (SCK.12 cells), wild-type SCK cells were transfected with a bicistronic expression plasmid, pWRG.mIL-12 (Dr. Ning-Sun Yang, Agracetus, Inc. Middleton, Wis.) that contains both the p35 and p40 subunit cDNAs of IL-12 under control of the cytomegalovirus (CMV) promoter, for production of bioactive mIL-12 (p70). Transfected cells were plated by limiting dilution and individual wells screened by p70 ELISA (Dr. David H. Presky, Hoffman-La Roche, Nutley, N.J.).

Clones were obtained that produce 1 or 12 ng mIL-12/10⁶ cells/24 hours (SCK.12A and C cells, respectively).

B. Cells Expressing IL-18

The IL-18 cDNA was obtained by RT-PCR, using RNA prepared from lipopolysaccharide (LPS)-induced total spleen RNA, based on the published sequence of mIL-18/IGIF (H. Okamura et al, cited above) using the primers: 5′(upper): GGCCCAGGAACAATGGCT SEQ ID NO: 1 and 3′ (lower): CCCTCCCCACCTAACTTTGAT SEQ ID NO: 2. An mIL-18 cDNA clone was sequenced to confirm normal coding potential and subcloned into the pLXSN retrovirus to create the viral vector pL(IL-18)SN. ψcre packaging cells were transfected by the calcium phosphate method and selected in G418 (400 μg/ml) to create resistant colonies that produce the L(mIL-18)SN retrovirus.

Supernatants from these cells were used to infect SCK cells in media containing 8 μg/ml polybrene. G418-resistant SCK clones (e.g. SCK.18A cells) were assayed for m/L-18 expression by Northern analysis and radioimmunoassay (RIA) as described in Example 2 to determine IL-18 expression.

To create SCK cells expressing higher levels of mIL-18 (e.g. SCK.18C cells), the mIL-18 cDNA was subcloned into the pEF2 vector which contains a neor gene (Gift of S. Pestka, UMDNJ, Piscataway, N.J.) and transfected into SCK cells by the calcium phosphate method. G418 resistant clones were analyzed by Northern analysis and RIA of Example 2 to determine expression. By Northern analysis, SCK.18C cells expressed significantly more recombinant mIL-18 mRNA than SCK.18A cells (data not shown).

### EXAMPLE 2

### IL-12, IFN-γ and IL-18 Radioimmunoassays

A. RIA of IL-12

IL-12 levels in the SCK.12 cell supernatants of Example 1 were determined by radioimmunoassay in duplicate for each sample as described previously (M. Wysocka et al, 1995 Eur. J. Immunol., 25:672-676). 24 hour supernatants were added to 96-well plates (Dynatech Laboratories) coated with 5 μg/ml C17.8 (anti-IL-12). After overnight incubation at 4° C., plates were washed with PBS-Tween-20. ¹²⁵I-labelled C17.8 was added to each well and incubated for 6 hours at 4° C. Bound ¹²⁵I-labelled antibody was assayed in a microplate scintillation counter (Topcount, Packard).

Blood samples were harvested by retroorbital eye bleeds from three mice per experimental group.

B. RIA for IFN-γ

mIFN-γ was determined for each sample by radioimmunoassay, as described previously (M. Wysocka et al, cited above). Serum samples were diluted 1:5 and added to 96-well plates (Dynatech Laboratories) coated with 5 μg/mL of AN18 mAb (anti-mIFN-γ). After overnight incubation at 4° C., plates were washed in PBS-Tween. ¹²⁵I-labelled XMG1.2 anti-mIFN-γ was added to each well and incubated for 6 hours at 4° C. Bound ¹²⁵I-labelled antibody was assayed in a microplate scintillation counter (Topcount, Packard).

C. RIA/ELISA for IL-18

IL-18 levels were determined by ELISA assay. To measure production of mIL-18/IGIF, a rabbit polyclonal antiserum specific for mIL-18 was generated by immunizing a rabbit with three doses of purified mIL-18 (100 μg/immunization; gift of R. Kastelein, DNAx Research Institute, Palo Alto, Calif.). The pre-bleed could not detect IL-18 in Western blots, nor did it neutralize the ability of IL-18 to stimulate NK cell production of IFN-γ. In contrast, the unfractionated antisera did detect rmIL-18 and mature mIL-18 in IFN-γ activated macrophages by Western blot. Furthermore, this antisera neutralized the ability of mIL-18 to enhance mIL-12-mediated production of IFN-γ by NK cells (data not shown).

A purified IgG fraction of the antisera was prepared (Harlan Bioscience) and used as the basis for a two site ELISA as previously described (J.

Abrams et al, 1992 Immunol. Rev., 127:5-24). The sensitivity of this assay is 300 pg/ml, and it did not detect IFN-γ, IL-12, IL-1a or IL-1b.

By radioimmunoassay SCK.18C cells secreted 0.51 ng mIL18/10⁶ cells/24 hrs while SCK.18A cells secreted less than 0.30 ng/10⁶ cells/24 hrs.

### EXAMPLE 3

### Tumor Formation by SCK Tumor Cells Expressing MIL-12 (SCK.12 Cells)

To determine if tumor cell secretion of IL-12 is effective in preventing tumor development in contrast to simply administering IL-12 to an animal which delays, but does not prevent, tumor development (see, C. Coughlin et al, cited above) the engineered SCK cells of Example 2A (SCK.12A and SCK.12C cells) and wild-type SCK cells were used.

The tumorigenesis studies were carried out using A/J or SCID mice. Female A/J mice, 6-8 weeks old, were purchased from The Jackson Laboratory (Bar Harbor, Me.). Female SCID mice, 6 weeks old, were bred at the Wistar Institute. All animals were maintained in microisolator cages and handled under aseptic conditions.

Cohorts of A/J mice (5-25 per group) were injected on day 0 with a dosage of the indicated tumor cells (column 1 of Table 1) subcutaneously (s.c.) in the flank. The cells were obtained from cultures established from low-passage, frozen stocks less than one week prior to injection, and the number of cells injected was based on the count of cells excluding trypan blue. Tumor formation was monitored daily.

Mice with tumors (second column) are indicated as the number of mice developing tumors/the number of mice in the cohort. The percent of mice developing tumors is indicated in parentheses. These results are compiled from six separate experiments.

The number of regressors is indicated in the third column of Table 1 by the number of tumors regressing/the number of tumors that developed in that group. Finally, the time to tumor is the number of days after the animals were injected with cells before the tumor became detectable. These data are expressed in the fourth column of Table 1 as the median number of days for all mice in the group that developed tumors plus or minus the standard deviation.

To test for immunity in mice that survived their initial exposure to transduced SCK cells, the survivors were challenged with 1×10⁵ SCK cells. Mice were monitored daily for tumor growth and progression. Euthanasia was performed according to Institutional Animal Care and Use Committee guidelines.

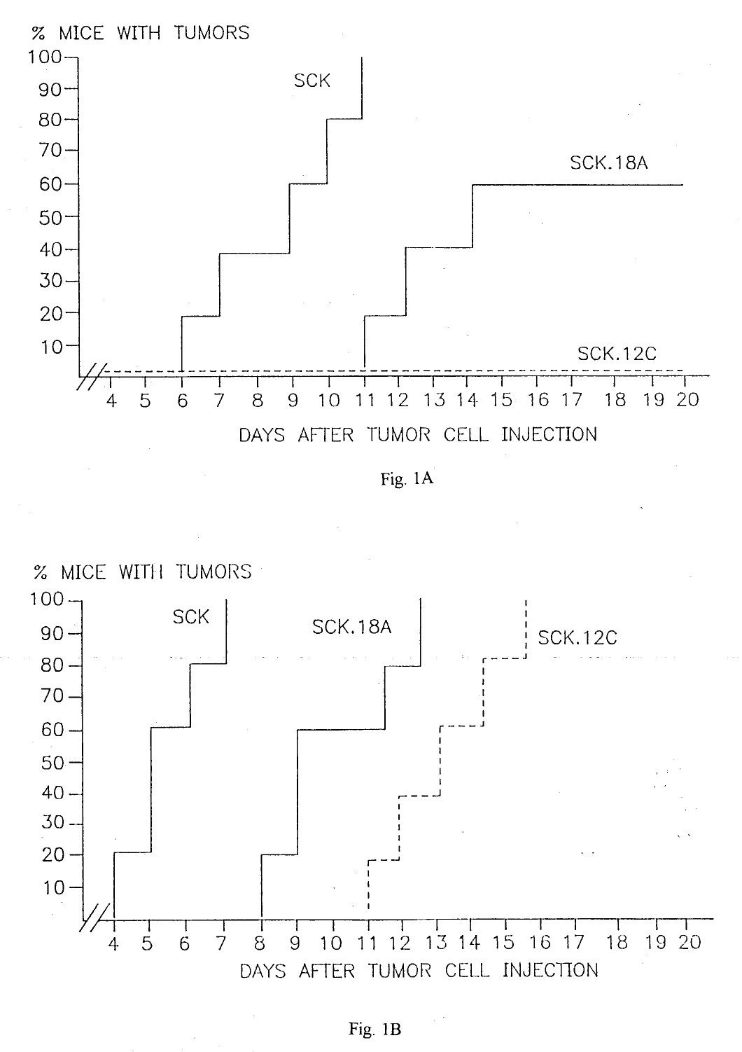

The results of this assay are shown in Table 1 above and FIG. 1A.

TABLE 1

| Tumor Cells Injected | Mice with Tumors | | Regressors | Time to Tumor |
|---|---|---|---|---|
| SCK | | | | |
| 2.5 × 10⁴ cells | 25/25 | (100%) | 0/25 | 7.5 ± 1.9 |
| SCK. 12A | | | | |
| 2.5 × 10⁴ cells | 17/22 | (77%) | 0/17 | 13.5 ± 4.1 |
| SCK. 12C | | | | |
| 2.5 × 10⁴ cells | 0/16 | (0%) | — | |
| 1.0 × 10⁶ cells | 3/5 | (60%) | 3/3 | 8.0 ± 0.6 |

Progressive tumors formed in 77% injected with SCK.12A cells, and in 0% injected with SCK.12C cells. SCK.12A tumors appeared about 6-8 days later than SCK tumors, which resembles the delay in SCK tumor appearance in mice treated with rmIL-12 (C. Coughin et al, cited above). SCK.12C cells did not form progressive tumors even when the dose of cells was increased to 1×10⁶ cells; 60% of these mice developed small tumors after about seven days that spontaneously regressed over the next three to four days. These data indicate that the reduced tumorigenicity of SCK cells secreting IL-12 depends on the level of secretion.

To determine whether the failure of SCK.12C cells to form progressive tumors was an intrinsic property of the cells or was due to a host response, 2.5×10⁴ SCK.12C cells were injected into SCID mice. All developed progressive tumors which appeared significantly later (about 7 days) than SCK tumors in SCID mice (FIG. 1B). These results indicate that T and/or B cells were required to prevent SCK.12C tumors, but that cells present in SCID mice, such as NK cells, were able to delay tumor formation.

### EXAMPLE 4

### Tumor Formation by SCK Tumor Cells Expressing MIL-18 (SCK.18 Cells)

To determine if local tumor cell secretion of mIL-18 can exert an antitumor effect, the SCK cells expressing mIL-18 (SCK.18A and SCK.18C) were employed. 2.5×10⁴ viable cells (SCK.18 or wild-type SCK cells) were injected subcutaneously (s.c.) in the flank of each A/J or SCID mouse.

Cohorts of A/J mice (5-25 per group) were injected on day 0 with a dosage of the indicated tumor cells (column 1 of Table 2) subcutaneously (s.c.) in the flank. The cells were obtained from cultures established from low-passage, frozen stocks less than one week prior to injection, and the number of cells injected was based on the count of cells excluding trypan blue. Tumor formation was monitored daily.

Mice with tumors (second column) are indicated as the number of mice developing tumors/the number of mice in the cohort. The percent of mice developing tumors is indicated in parentheses. These results are compiled from six separate experiments.

The number of regressors is indicated in the third column of Table 2 by the number of tumors regressing/the number of tumors that developed in that group. Finally, the time to tumor is the number of days after the animals were injected with cells before the tumor became detectable. These data are expressed in the fourth column of Table 2 as the median number of days for all mice in the group that developed tumors plus or minus the standard deviation.

To test for immunity in mice that survived their initial exposure to transduced SCK cells, the survivors were challenged with 1×10⁵ SCK cells. Mice were monitored daily for tumor growth and progression. Euthanasia was performed according to Institutional Animal Care and Use Committee guidelines.

The results for A/J mice are seen in FIG. 1A and Table 2.

TABLE 2

| Tumor Cells Injected | Mice with Tumors | | Regressors | Time to Tumor |
|---|---|---|---|---|
| SCK | | | | |
| 2.5 × 10⁴ cells | 25/25 | (100%) | 0/25 | 7.5 ± 1.9 |
| SCK. 18A | | | | |
| 2.5 × 10⁴ cells | 19/28 | (68%) | 0/19 | 13.0 ± 3.1 |
| SCK. 18C | | | | |
| 2.5 × 10⁴ cells | 3/10 | (30%) | 0/3 | 17.0 ± 2.1 |
| 1 × 10⁵ cells | 4/5 | (80%) | 0/4 | 13.5 ± 1.7 |
| 1 × 10⁶ cells | 5/5 | (100%) | 0/5 | 12.0 ± 2.3 |

Both SCK.18A and SCK.18C cells behaved like SCK cells in vitro, but when A/J mice were injected with 2.5×10⁴ SCK.18A or SCK.18C cells, 68% and 30% of mice developed tumors, respectively (Table 2). SCK.18 tumors were delayed compared to SCK tumors, and SCK.18C tumors developed more slowly than SCK.18A tumors (FIG. 1A and Table 2).

While these data show that SCK.18C cells are more tumorigenic than SCK.12C cells (see Example 3), no conclusions can be drawn about the comparative antitumor potency of secreted mIL-12 and mIL-18 because a plateau of effectiveness was never reached.

The SCID mice injected with SCK.18A cells uniformly developed tumors but their appearance was delayed compared to SCK tumors (FIG. 1B). Thus, T and/or B cells were required for rejection of SCK.18A cells, but cells present in SCID mice were able to delay tumor formation.

### EXAMPLE 5

### Histology of Cytokine-Producing Cells

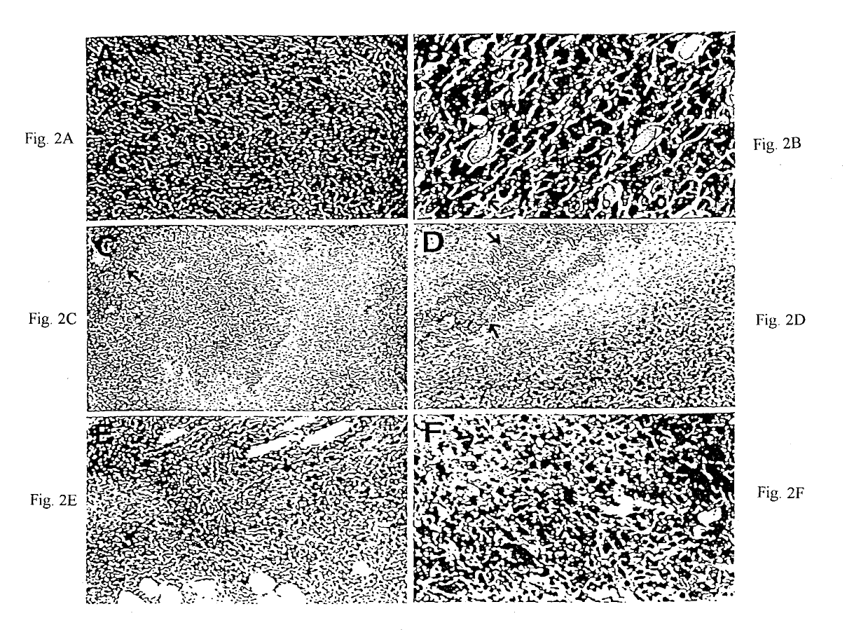

A/J mice were injected with 2.5×10⁴ SCK cells, including the IL-12 and IL-18 expressing SCK cells. Four days after tumors became palpable, they were removed, fixed in 10% buffered formalin and embedded in paraffin. Paraffin sections were stained with hematoxylin/eosin. Histologic examination of SCK.12 and SCK.18 tumors revealed that, unlike SCK tumors which have only single cells undergoing cell death (FIG. 2A, FIG. 2B indicated by arrows), both mIL-12 and mIL-18-expressing tumors have significant areas of necrosis by day four (FIGS. 2C, 2D and 2E, 2F, respectively). While SCK.18 tumors demonstrate focally necrotic areas by day four (indicated by arrows, FIG. 2E), SCK.12 tumors have significantly more extensive necrosis with only scattered areas of viable tumor cells (indicated by arrow, FIG. 2C).

Both SCK.12 and SCK.18 cells induce significant inflammatory response within areas of necrosis consisting primarily of polymorphonuclear cells (arrows, FIG. 2D, F) which is not seen in SCK tumors (FIG. 2A). Together, these observations indicate that mIL-18 and mIL-12 induce significantly more inflammation than SCK tumors alone and that mechanisms inducing tumor cell death occur more rapidly in SCK.12 and SCK.18 tumors than in the wild-type SCK tumors.

### EXAMPLE 6

### Antibody Neutralization of IL-12 or IL-18

To determine if the cytokine-stimulated production of IFN-γ was responsible for the observed antitumor activity of Examples 3 and 4, antibody neutralization studies were performed substantially as follows:

A. IL-12

Tumor cells of SCK.12C were injected subcutaneously in the flank region of the mouse. The dose of cells is 2.5×10⁴ cells/mouse (column one of Table 3, one experiment). In vivo neutralization of IFN-γ or IL-12 was accomplished by injecting A/J mice with either monoclonal anti-IFN-γ antibody (XMG.6, gift from Alan Sher, NIH) (M. Wysocka et al, cited above) or monoclonal anti-IL-12 antibody (C17.15) at 0.5 mg/injection/mouse on days −1, +1, +3, and +6. Normal rat antibody (0.5 mg/injection/mouse) or PBS was injected into control mice on the same schedule.

The ability of the anti-IFN-γ and anti-IL-12 monoclonal antibodies to deplete mice of IFN-γ or IL-12 was previously demonstrated (M. Wysocka et al, cited above). The results of this experiment are reported in Table 3 below. The third column indicates the number of days after the animal was injected with cells before the tumor became detectable. These data are expressed in the fourth column of Table 3 as the median number of days for all mice in the group that developed tumors plus or minus the standard deviation.

TABLE 3

| Tumor Cells Injected | Antibody | Mice with Tumors | Time to Tumor |
|---|---|---|---|
| SCK. 12C | None | 0/5 | — |
| SCK. 12C | NRA | 0/5 | — |
| SCK. 12C | α-IFN-γ | 4/5 | 8.0 ± 1.8 |
| SCK. 12C | α-IL-12 | 5/5 | 10.5 ± 2.9 |

Following injections of anti-IFN-γ antibody (XMG.6) to mice injected with SCK.12C cells, 4/5 developed progressive tumors. This resembled the uniform development of SCK.12C tumors in mice given anti-mIL-12 antibody and contrasted with the lack of tumors in mice given control or no antibody. Importantly, treatment of mice with anti-IFN-γ antibody abrogated the delay in tumor development normally seen with SCK.12 cells (Table 3) and with rmIL-12 therapy (C. Coughlin et al, cited above) suggesting that IFN-γ mediates the delay in tumor development and plays a crucial role in the antitumor protection.

B. IL-18

The experiment reported above was repeated substantially similarly using the IL-18 expressing SCK cells and the results are reported in Table 4.

TABLE 4

| Tumor Cells Injected | Antibody | Mice with Tumors | Time to Tumor |
|---|---|---|---|
| SCK. 18C | None | 1/5 | 17 |
| SCK. 18C | NRA | 2/5 | 16.0 ± 2.8 |
| SCK. 18C | α-IFN-γ | 5/5 | 7.0 ± 16 |
| SCK. 18C | α-IL-12 | 2/5 | 15.0 ± 2.8 |

When A/J mice were given anti-IFN-γ antibody, 5/5 mice given SCK.18C cells rapidly developed tumors (Table 4). Since tumors only developed in 1/5 mice given no antibody and 2/5 mice given control antibody, IFN-γ is required both for tumor rejection and the delay in tumor development in tumors secreting mIL-18.

Interestingly, the mIL-18 antitumor effect did not require endogenous mIL-12 because only 2/5 mice given anti-mIL-12 antibody developed tumors, which were delayed in their appearance. These antibody neutralization studies revealed that the antitumor effects of secreted IL-12 and IL-18 required IFN-Y and that the antitumor effects of IL-18 did not require mIL-12.

### EXAMPLE 7

### SCK.12 and SCK.18 Cells Synergistically Induce Systemic Protection

A. To test whether SCK12C cells, which demonstrated a striking absence of tumor formation, could induce rejection of SCK cells, a group of A/J mice (n=25) were injected with 2.5×10⁴ SCK cells in the right or left flank, a second group of mice (n=10) were injected with 2.5×10⁴ SCK.12C cells in the right flank and 2.5×10⁴ SCK cells in the same location (by mixing the two cell types in vitro prior to injection)- and a third group (n=0) received the same amounts of SCK and SCK.12C cells in opposite flanks. All cells were injected on day 0, and mice were monitored daily for tumor development.

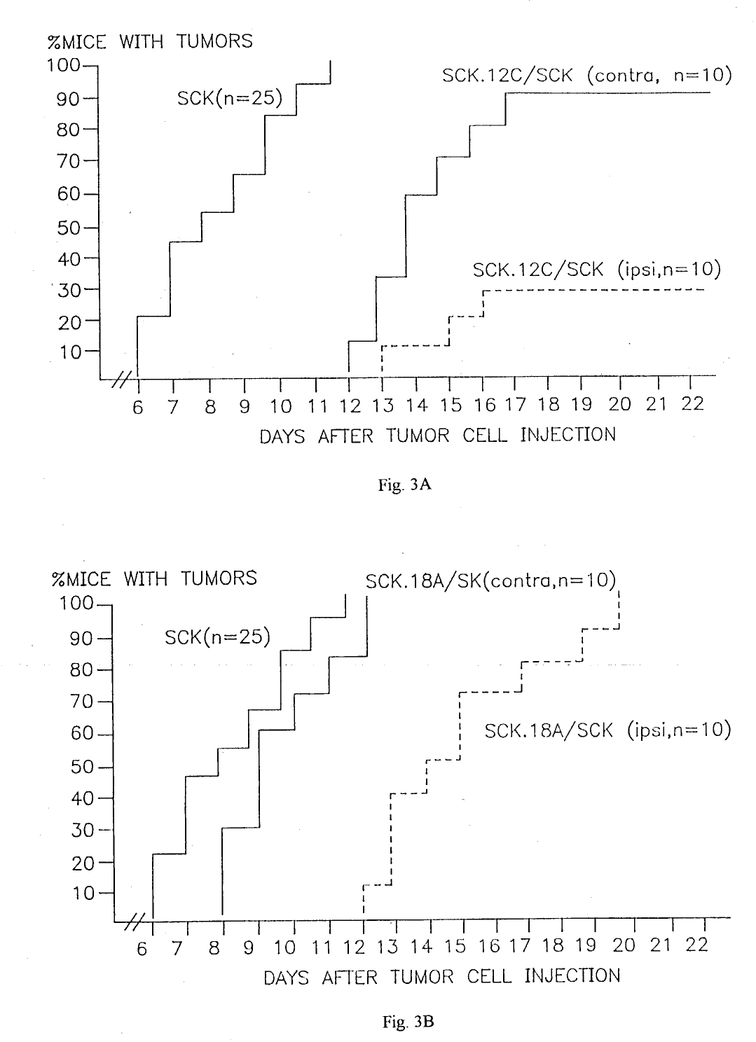

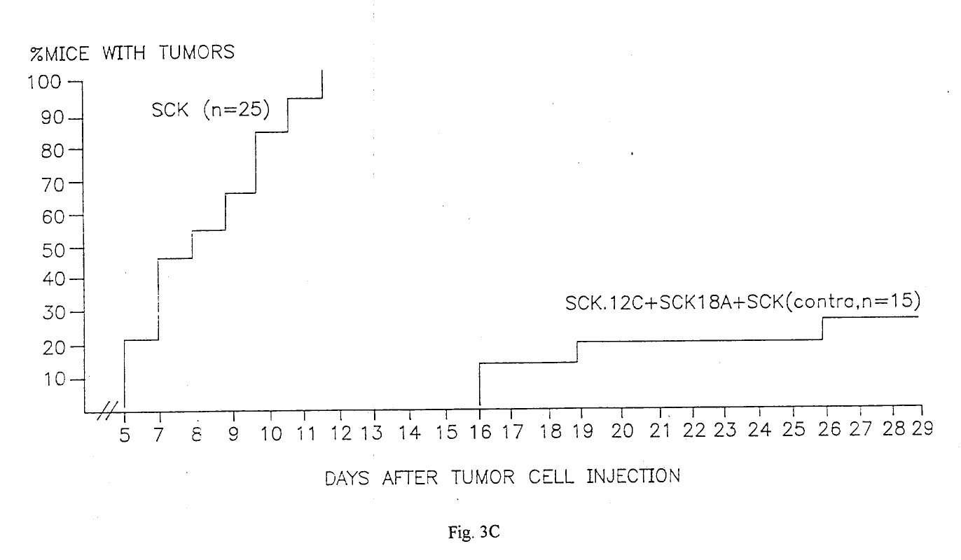

The results shown in Table 5 below and FIG. 3A are compiled from two separate experiments, with consistent results. The third column indicates the number of mice developing tumors/the number of mice in the cohort. The percent of mice developing tumors is indicated in parentheses. Time to tumor is the number of days after the animal was injected with cells before the tumor became detectable. These data are expressed in the fourth column of Table 5 as the median number of days for all mice in the group that developed tumors plus or minus the standard deviation.

The results indicate that in mice co-injected with SCK and SCK.12C cells at the same site, only 30% developed tumors, and these were delayed in appearance. When SCK cells were injected at a distance from the SCK.12C cells, 90% of mice developed SCK tumors which were delayed in their appearance.

B. In another experiment conducted substantially as described above, one group of mice (n=25) received 2.5×10⁴ SCK cells alone; a second group (n=10) received SCK.18A and SCK cells co-injected in one flank; a third group (n=10) received SCK and SCK.18A cells in opposite flanks. A similar experiment was performed using SCK.18C cells.

As expected from the weak antitumor activity of SCK.18A cells alone, protection from SCK tumors was indeed poor (Table 5 and FIG. 3B). All mice co-injected with SCK mixed with SCK.18A cells developed tumors (which were delayed in appearance); all mice injected with SCK.18A and SCK cells in opposite flanks developed SCK tumors with only a slight delay, and 8/10 also developed SCK.18A tumors. Although SCK.18C cells secrete more mIL-18 and are less tumorigenic than SCK.18A cells, a coinjection experiment performed with SCK.18C cells produced similar results (data not shown). These data indicate that the protection conferred by SCK.12C cells has strong local effects, but cannot induce systemic protection against SCK tumor development.

C. To test whether mIL-12 and mIL-18 together might produce a synergistic antitumor response, in view of their ability to synergistically induce IFN-γ by T cells in vitro (M. Micallef et al, cited above), in another experiment, one group of mice (n=25) received 2.5×10⁴ SCK cells alone; and a second group (n=15) were injected with a mixture of 2.5×10⁴ SCK.12C cells and 2.5×10⁴ SCK.18A cells in the right flank and 2.5×10⁴ SCK cells in the left flank.

Although neither SCK.12C nor SCK.18A cells alone provided mice with much protection, the two cell types together protected the majority of mice from distant SCK tumors which were markedly delayed when they developed (FIG. 3C and Table 5). The difference in survival between mice receiving SCK.12C and SCK.18A cells (70%) and mice receiving either cell type alone (10% for SCK.12C, 0% for SCK.18A) is highly significant (p<0.005) and indicates a synergistic or cooperative induction of systemic tumor protection by the two secreted cytokines.

TABLE 5

| Tumor Cells Injected | | Mice | | |
|---|---|---|---|---|
| Right Flank | Left Flank | with Tumors | | Time to Tumor |
| SCK | SCK | 10/10 | (100%) | 8.5 ± 2.3 |
| SCK. 12C | None | 0/10 | (0%) | — |
| SCK. 12C + SCK | None | 3/10 | (30%) | 15.0 ± 1.5 |
| SCK. 12C | SCK | 9/10 | (90%) | 14.0 ± 1.6 |
| SCK. 18A | None | 7/10 | (70%) | 13.0 ± 3.1 |
| SCK. 18A + SCK | None | 10/10 | (100%) | 14.5 ± 2.7 |
| SCK. 18A | SCK | 10/10 | (100%) | 9.0 ± 1.6 |
| SCK. 12C + SCK. 18A | SCK | 3/10 | (30%) | 19.0 ± 5.1 |

D. Another experiment demonstrated that this protection was dependent on endogenously produced IFN-γ. Tumor cells of the indicated type were injected subcutaneously in the indicated flank. When two cell lines were injected into the right flank, they were mixed in vitro prior to injection. The dose of cells was 2.5×10⁴ cells/cell line/mouse. These results are compiled from two separate experiments with consistent results. Antibodies of the indicated type were injected on days −1, +1, +3, and +6 with respect to tumor cell injections (day 0). Mice were treated with anti-IFN-γ antibody, which abrogated protection.

The data is shown in Table 6 below. “Mice with tumors” is indicated by the number of mice developing tumors/the number of mice in the cohort. Time to tumor is the number of days after the animal was injected with cells before the tumor became detectable. These data are expressed in the fourth column of Table 6 as the median number of days for all mice in the group that developed tumors plus or minus the standard deviation.

In this particular antibody ablation experiment, 2/5 mice given no antibody and 3/5 mice given control antibody developed tumors, with the characteristic delay in tumor appearance.

TABLE 6

| Tumor Cells Injected | | | Mice with | Time to |
|---|---|---|---|---|
| Right Flank | Left Flank | Antibody | Tumors | Tumor |
| SCK. 12C + SCK. 18A | SCK | None | 2/5 | 22.0 ± 4.2 |
| SCK. 12C + SCK. 18A | SCK | NRA | 3/5 | 22.0 ± 5.5 |
| SCK. 12C + SCK. 18A | SCK | α-IFN-γ | 5/5 | 8.0 ± 1.1 |

E. To test whether the systemic protection afforded by the combination of SCK.12C+SCK.18A cells could protect against previously established SCK tumors, mice were injected with SCK cells in the left flank on day 0 and with SCK.12C+SCK.18A cells in the right flank on day 3.

Whereas neither SCK.12C nor SCK.18A cells alone induced rejection of these established SCK tumors (SCK.12C cells did delay their growth), the combination induced rejection in 3/10 mice. This showed that the combination of secreted IL-12 and IL-18 could control established SCK tumors which is remarkable because SCK tumors are very aggressive. Their rapid clinical course offers a very narrow window of therapeutic opportunity and, previously with other maneuvers, the inventors have been unable to cure A/J mice of SCK tumors established three days earlier (C. Coughlin et al, cited above).

Taking the results of Examples 3 through 7 together, in summary, in syngeneic A/J mice, SCK cells expressing IL-12 or IL-18 were less tumorigenic and formed tumors more slowly in a cytokine-dose dependent manner; whereas in severe combined immunodeficient (SCID) mice, in the presence of the two cytokines, tumors were consistently formed, but more slowly than control cells.

Cancer cells expressing the most IL-12, SCK.12C cells, were the least tumorigenic and only formed spontaneously regressing tumors when inocula 40 times the usual size were injected. SCK.12C cells protected 70% of mice against tumorigenesis by SCK cells injected at the same site, but protected only 10% of mice against tumorigenesis by SCK cells at a distant site.

While inoculation of SCK.12 or SCK.18 cells alone offered minimal systemic protection against SCK tumors, surprisingly inoculation of the two cell types together synergistically protected 70% of mice from concurrently injected distant SCK cells and 30% of mice from SCK cells established three days earlier.

### EXAMPLE 8

### Antitumor Immunity Induced by SCK.12 and SCK.18 Cells

Antitumor immunity in mice that rejected SCK.12C and/or SCK.18A cells was examined by SCK rechallenge studies.

A. To assess whether the mechanism by which SCK.12+SCK.18 cells protect against SCK tumors is due to antigen-specific immune responses induced against the tumor cells, mice that survived injections of the tumor cells indicated at a dose of 2.5×10⁴ cells/cell line/mouse (column 1, Table 7) were rechallenged with 1×10⁵ parental SCK cells (four times the usual challenge dose) on the indicated number or days after the initial injections of cells (day 0) (column 2, Table 7). The third column reports the number of mice developing tumors/the number of survivors rechallenged in the cohort. Survival is the percent of rechallenged mice in the group that rejected the rechallenge dose of SCK cells.

As illustrated in Table 7 below, of the 16 mice that survived an initial challenge of 2.5×10⁴ SCK.12C cells, seven (44%) rejected this rechallenge, indicating that less than half of the survivors had protective immunity.

Of eight survivors of 2.5×10⁴ SCK.18A cells, four (50%) rejected their rechallenge, and of 13 survivors of 2.5×10⁴ SCK.12C+SCK.18A cells, six (46%) rejected their rechallenge.

Thus, approximately half or less of the mice surviving any of these challenges were protected two months later, which is low considering the absence of SCK.12C tumorigenicity and the excellent systemic protection afforded by SCK.12C+SCK.18A cells.

B. To test whether protective immunity had been present but was short—lived and had waned by two months, mice that were tumor-free after injection of SCK.12C or SCK.12C+SCK.18A cells were rechallenged in the opposite flank with 1×10⁵ SCK cells two or four weeks after their initial challenge.

At two weeks, 1/8 mice given SCK.12C cells alone and 4/8 mice given SCK.12C+SCK.18A cells survived their rechallenge, while at four weeks, 4/8 mice given SCK.12C cells alone were protected (Table 7). Clearly, mice given SCK.12C cells with or without SCK.18A cells were not better protected at two weeks than at two months.

This rechallenge data indicated that protective immunity did not develop quickly or consistently in mice given SCK.12C cells. Half or fewer of these mice had protective immunity and that protective immunity took more than two weeks to develop in mice exposed to SCK.12C cells alone. Antitumor immunity therefore could not account for the observed reduction in SCK.12C tumorigenicity and was unlikely, by itself, to underlie the systemic protection afforded by co-injected SCK.12C+SCK.18A cells. That SCK.12C cells protected well against local but not distant SCK tumorigenesis reinforced this conclusion.

TABLE 7

| Mice Surviving Injected | Day of | Mice Surviving | |
|---|---|---|---|
| Tumor Cells | Rechallenge | Rechallenge | Survival |
| SCK 12.C | 14 | 1/8 | 12% |
| | 28 | 4/8 | 50% |
| | 60 | 7/16 | 44% |
| SCK. 18A | 60 | 4/8 | 50% |
| SCK. 12C + SCK. 18A | 14 | 4/8 | 50% |
| | 60 | 6/13 | 46% |

### EXAMPLE 9

### Cytotoxic T Lymphocyte (CTL) Assays

The spleens of the mice treated in the above examples were examined for evidence of SCK-specific cytolytic activity as follows:

Spleens of A/J mice injected with SCK.12C, SCK.18A or SCK.12C+SCK.18A cells were removed. Red blood cell depleted spleen cell suspensions were cocultured at 4×10⁶ cells with 1×10⁵ γ-irradiated (20,000 rads) SCK or HKB cells per well in 24-well plates in 2 mls of RPMI 1640 supplemented with 5% heat inactivated fetal calf serum (FCS), 2-mercaptoethanol, 10 mM HEPES pH 7.9, sodium pyruvate, non-essential amino acids, and penicillin/streptomycin. Cultures were incubated for five days.

For the determination of cytolytic activity, 106 SCK (NK-resistant targets) or YAC cells (NK susceptible targets) were labeled with 100 μCi ⁵¹Cr for 90 minutes and washed three times in PBS. Effector cultures were harvested, washed three times with PBS and coincubated with 5000 labeled target cells at decreasing ratios (starting at 50:1). After 8 hours, chromium release was measured in 100 μl supernatant from each well. Specific lysis was calculated as follows: % Specific Lysis=100×(mean experimental CPM−mean spontaneous CPM)/(mean maximum CPM−mean spontaneous CPM).

Splenocytes from SCK-vaccinated or immune mice restimulated with control HKB cells consistently generate cultures with background levels of SCK cytolytic activity.

Splenocytes from A/J mice injected with SCK.12C, SCK.18A or SCK.12C+SCK.18A cells as described in Example 8 fourteen days earlier had no significant CTL activity.

### EXAMPLE 10

### Inhibition of Angiogenesis Mediated by SCK.12 and SCK.18 Cells

To examine the role of angiogenesis inhibition, Matrigel® implants containing SCK cells or another angiogenic stimulus were examined. Very large inocula of SCK.12C cells (40 times the usual number of cells injected) formed only small tumors that spontaneously regressed. Further, it is known that administration of rmIL-12 can inhibit angiogenesis (E. Voerst el al, cited above and C. Sgadari et al, cited above).

To test the hypothesis that inhibition of tumor angiogenesis underlies the behavior and effects of SCK.12C and SCK.18A cells, Matrigel® (Collaborative Biomedical Products, non-growth factor reduced) implants were employed.

Matrigel® implants are formed from a solution of basement membrane components derived from murine EHS sarcoma cells (H. Kleinman et al, 1982 Biochemistry, 21:6188-6193). Matrigel® implant material injected subcutaneously into mice forms a solid implant that supports new vessel growth if an angiogenic stimulus is present.

Thus, in vivo assays for tumor angiogenesis were carried out by injecting A/J mice with 0.5 ml Matrigel® implant solution mixed on ice with either 10 ng recombinant basic FGF (b-FGF) or 1×10⁵ SCK, SCK.12 or SCK.18 cells as the angiogenic stimulus. The Matrigel® implant was injected subcutaneously in the abdominal midline on day 0 in all experiments. Recombinant mIL-12 was injected, where indicated, on day −1, 0, 1, 2 and 3. Monoclonal anti-IFN-γ antibody (XMG.6) was injected on days −1, +1, and +3. Matrigel® implant plugs were harvested on day 4 and photographed using a dissecting microscope.

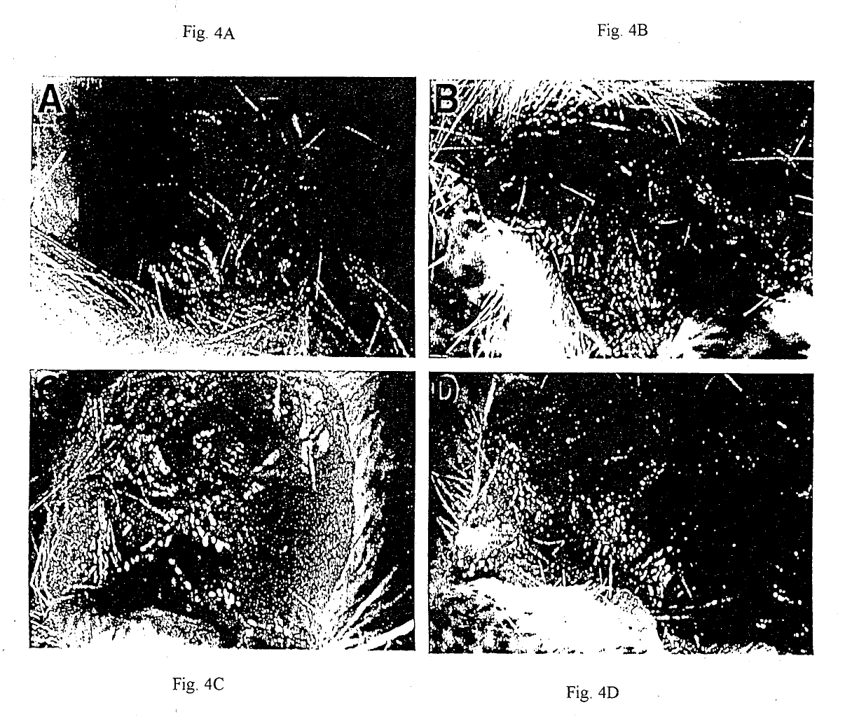

The photographic results are displayed in FIGS. 4A-4D. Matrigel® (0.5 ml) implants without additives are pale and unvascularized four days after implantation (FIG. 4A). Inclusion of 10 ng rb-FGF or 1×10⁵ SCK cells in the implant provides an angiogenic stimulus that makes it orange colored and visibly vascularized (FIG. 4B shows an implant containing 1×10⁵ SCK cells). SCK.12C and SCK.18A cells in Matrigel® implants (FIGS. 4C and 4D, respectively) do not induce nearly the same degree of vascularization as SCK cells.

To quantitate Matrigel® implant vascularization, their hemoglobin content was measured six days after implantation to assess the number of vessels supporting blood circulation. Quantitation of hemoglobin was performed by the Drabkin method, as described previously (A. Passaniti et al, 1992 Lab. Invest., 67:519-528). Briefly, Matrigel® implant pellets were harvested from mice treated as above after six days and all surrounding tissue was dissected away. Pellets were melted at 4° C., and assayed for hemoglobin content using the Drabkin method (Drabkin's reagent kit, Sigma Diagnostics).

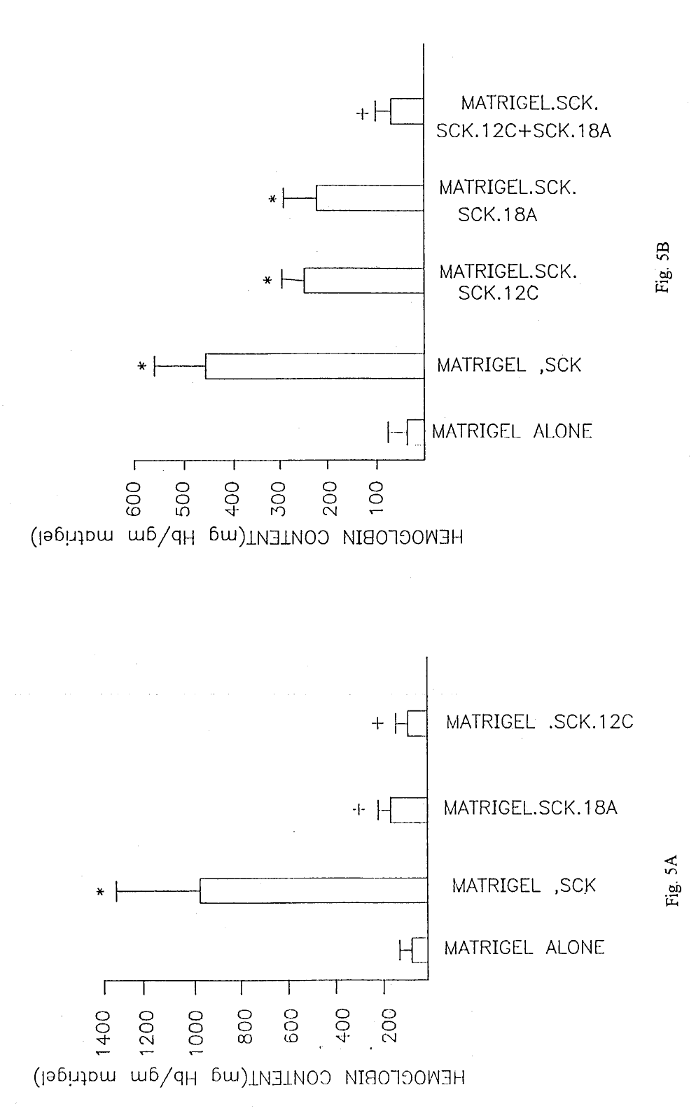

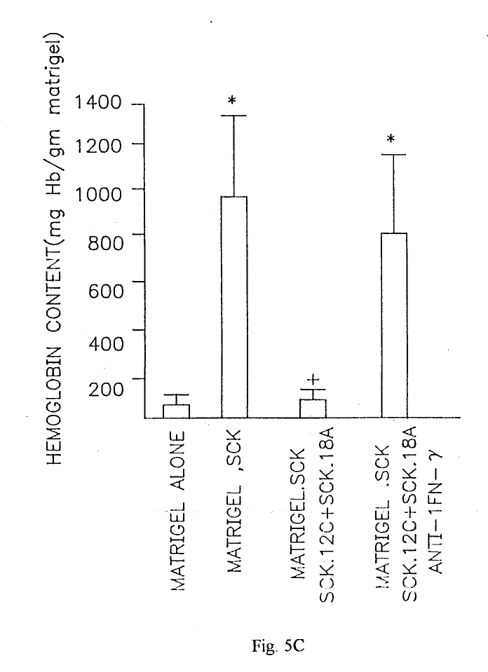

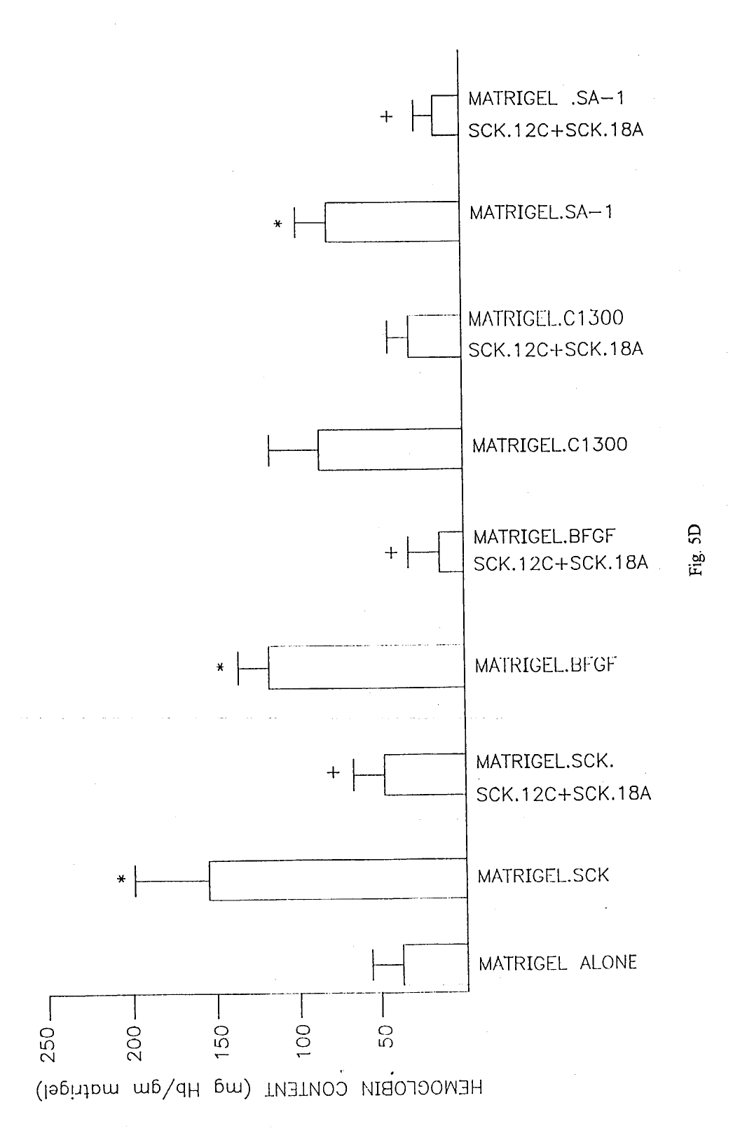

The results of the hemoglobin quantitation are illustrated in FIGS. 5A-5D. FIG. 5A displays the hemoglobin content of different Matrigel® implants in an experiment similar to that shown in FIG. 4 and reveals that SCK.12C and SCK.18A cells induced much less vascularization than SCK cells. This could result from decreased production of angiogenic factors by the engineered tumor cells and/or from the presence of an angiogenesis inhibitor. An inhibitor is present at the least, inasmuch as Matrigel® implants containing SCK.12C cells or an equal mixture of SCK and SCK.12C cells were equally poorly hemoglobinized (data not shown). Inhibition of tumor angiogenesis by SCK.12C cells may explain why these cells are essentially nontumorigenic and can effectively prevent tumorigenesis by co-injected SCK cells.

Both SCK.12C and SCK.18A cells reduced angiogenesis of distant Matrigel® implants containing SCK cells, but SCK.12C+SCK.18A cells together inhibited systemic angiogenesis more effectively than either cell type alone (FIG. 5B). This cooperative effect might contribute to or be responsible for the better protection against distant SCK tumors afforded by the combination of mIL-12 and mIL-18-secreting cells. The systemic antiangiogenic effect of SCK.12C+SCK.18A cells is mediated by IFN-γ because inhibition of angiogenesis by these cells was abrogated by antibody neutralization of IFN-γ (FIG. 5C). This is not surprising considering that IFN-γ has been shown to mediate the antiangiogenic effect of recombinant mIL-12 (E. Voerst et al, cited above and C. Sgadari et al, cited above) and both mIL-12 and mIL-18 induce IFN-γ production.

Angiogenesis inhibition by induced secretion of IFN-γ indicates that the antiangiogenic effect of SCK.12C+SCK.18A cells is not tumor cell or angiogenic factor specific. The ability to inhibit angiogenesis of implants containing rb-FGF or other syngeneic tumor cells was tested. The combination of mIL-12 and mIL-18 secreted by SCK cells effectively inhibited angiogenesis induced by rb-FGF, C1300 neuroblastoma cells or Sa-1 sarcoma cells (FIG. 5D; in this particular experiment, the level of implant hemoglobinization was lower that in previous experiments). Thus, the antiangiogenic effects of tumor cell-secreted mIL-12 and mIL-18 are not limited to angiogenesis induced by homologous tumor cells and are active against different stimulants of neovascularization. That this contributes to the antitumor effects of SCK.12C+SCK.18A cells was shown by the fact that these cells could occasionally prevent and consistently retard tumorigenesis by distant Sa-1 cells (data not shown).

In summary, while Matrigel® implant neovascularization was significantly inhibited by both SCK.12C and SCK.18A cells, the two cell types together produced significantly greater systemic inhibition of angiogenesis. Inhibition was dependent on IFN-γ and occurred whether SCK, unrelated syngeneic Sa-1 or C1300 tumor cells, or basic FGF provided the angiogenic stimulus.

All published documents are incorporated by reference herein. Numerous modifications and variations of the present invention are included in the above-identified specification and are expected to be obvious to one of skill in the art. Such modifications and alterations to the compositions and processes of the present invention are believed to be encompassed in the scope of the claims appended hereto.

What is claimed is:

1. A method for treating a mammal having a tumor by adaptive immunotherapy comprising co-administering to a mammal an engineered mammalian cell that expresses IL-12 in vivo and an engineered mammalian cell that expresses IL-18 in vivo, wherein said co-expression of said IL-12 and IL-18 induces a systemic and synergistic anti-tumor response.

2. The method according to claim 1, wherein said co-administration comprises concurrently administering said cell that expresses IL-18 and said cell that expresses IL-12 in a single composition.

3. The method according to claim 1, wherein said IL-18 and IL-12 are co-expressed in the same cell.

4. The method according to claim 1, wherein said co-administration comprises sequentially administering said cell that expresses IL-18 and said cell that expresses IL-12 in any order.

5. The method according to claim 1 wherein said engineered cells that express said IL-12 and IL-18 are tumor cells.

6. The method according to claim 5, wherein said engineered tumor cells are of the same tumor type as the tumor of said mammal.

7. The method according to claim 1, wherein said engineered cells are admininstered to said mammal in a suitable pharmaceutical carrier.

8. The method according to claim 7, wherein said carrier is a biocompatible implant.

9. The method according to claim 1, wherein said adminstration is subcutaneous or intramuscular.

10. The method according to claim 1, wherein said administration is made at a site in said mammal proximal to said tumor.

11. The method according to claim 1, wherein said engineered cell comprises regulatory sequences that direct expression of said IL-12 or L-18 therein.

12. The method according to claim 1, wherein said anti-tumor response is an immune response.

13. The method according to claim 1, wherein said anti-tumor response comprises a retardation in the growth of said tumor.

14 A pharmaceutical composition for adaptive immunotherapy comprising a pharmaceutically acceptable carrier and at least one of the group consisting of an engineered mammalian cell that expresses and secretes IL-12 in vivo, an engineered mammalian cell that expresses and secretes IL-18 in vivo and an engineered mammalian cell that expresses and secretes IL-12 and IL-18 in vivo.

15. The composition according to claim 14, wherein said engineered cell comprises at least one regulatory sequence that directs expression of said IL-12, said IL-18, or a combination thereof.

16. The composition according to claim 14 wherein said carrier in a biocompatible implant.

17. The composition according to claim 14 wherein said cell is a tumor cell.

18. The composition according to claim 17, wherein said tumor cell is a cell removed from said mammal's tumor and transfected with a nucleic acid sequence comprising a sequence encoding IL-12, IL-18 or the combination thereof and at least one regulatory sequence directing expression thereof in said cell.

19. The composition according to claim 14 comprising a first engineered mammalian cell that expresses and secretes IL-12 in vivo and a second engineered mammalian cell that expresses and secretes IL-18 in vivo.

20. The composition according to claim 14 comprising an engineered mammalian cell that expresses and secretes IL-12 and IL-18 in vivo

21. The composition according to claim 20, wherein said cell contains a bicistronic nucleic acid sequence that encodes expression of IL-12 and IL-18 under at least one regulatory sequence.

22. The composition according to claim 20, wherein said cell contains a first nucleic acid sequence encoding expression of IL-12 under control of a regulatory sequence and a second nucleic acid sequence encoding expression of IL-18 under control of a second regulatory sequence, said second sequence independent of said first sequence.
